SEQUENCE LISTING (21 sequences)

SEQ ID NO: 1 - DNA, 23 bp, Bovine
ccttttcctctagcatcaagtta

SEQ ID NO: 2 - DNA, 23 bp, Bovine
ccttttcctctagcatcaagtta

SEQ ID NO: 3 - DNA, 25 bp, Artificial Sequence
  Zipcode oligonucleotide
gatgatcgacgagacactctcgcca

SEQ ID NO: 4 - DNA, 25 bp, Artificial Sequence
  Zipcode oligonucleotide
cggtcgacgagctgccgcgcaagat

SEQ ID NO: 5 - DNA, 25 bp, Artificial Sequence
  Zipcode oligonucleotide
gacattcgcgatcgccgcccgcttt

SEQ ID NO: 6 - DNA, 46 bp, Artificial Sequence
  Capture oligonucleotide
tggcgagagtgtctcgtcgatcatccatcaagttaacacgtggagc

SEQ ID NO: 7 - DNA, 46 bp, Artificial Sequence
  Capture oligonucleotide
atcttgcgcggcagctcgtcgaccgcatcaagttaacacgtggagg

SEQ ID NO: 8 - DNA, 46 bp, Artificial Sequence
  Capture oligonucleotide
aaagcgggcggcgatcgcgaatgtccatcaagttaacacgtggagw

SEQ ID NO: 9 - DNA, 42 bp, Artificial Sequence
  Signalcode reporter oligonucleotide
acattccccagtttaatactgcgtcaagatgctaccgttcag

SEQ ID NO: 10 - DNA, 22 bp, Bovine
acattccccagtttaatactgc

SEQ ID NO: 11 - DNA, 20 bp, Artificial Sequence
  Anti-Signalcode oligonucleotide
ctgaacggtagcatcttgac

SEQ ID NO: 12 - DNA, 24 bp, Bovine
aatagtcattttgtccaacctcta

SEQ ID NO: 13 - DNA, 24 bp, Bovine
cctaagcattttaggtgagataca

SEQ ID NO: 14 - DNA, 25 bp, Artificial Sequence
  Zipcode oligonucleotide
cttttcccgtccgtcatcgctcaag

SEQ ID NO: 15 - DNA, 25 bp, Artificial Sequence
  Zipcode oligonucleotide
cttttcccgtccgtcatcgctcaag

SEQ ID NO: 16 - DNA, 25 bp, Artificial Sequence
  Zipcode oligonucleotide
ggctgggtctacagatccccaactt

SEQ ID NO: 17 - DNA, 48 bp, Artificial Sequence
  Capture oligonucleotide
gtggtccatcacaaacagggagtcgcaggtaggaaatttgaaatgtta

SEQ ID NO: 18 - DNA, 48 bp, Artificial Sequence
  Capture oligonucleotide
cttgagcgatgacggacgggaaaagcaggtaggaaatttgaaatgttg

SEQ ID NO: 19 - DNA, 48 bp, Artificial Sequence
  Capture oligonucleotide
aagttggggatctgtagacccagcccaggtaggaaatttgaaatgtty

SEQ ID NO: 20 - DNA, 46 bp, Artificial Sequence
  Signalcode oligonucleotide
caagattaaacttttaaagtcacatggtcaagatgctaccgttcag

SEQ ID NO: 21 - DNA, 26 bp, Bovine
caagattaaacttttaaagtcacatg

(19) United States
(12) Patent Application Publication
(10) Pub. No.: US 2005/0009015 A1
(43) Pub. Date: Jan. 13, 2005

(54) Method for relative quantification of attached nucleic acids

(76) Inventors: Wan Ji, Austin, TX (US); Keqin Gregg, Austin, TX (US); Bonnie Reus, Cedar Park, TX (US); Jon Kemppainen, Austin, TX (US); Scott Davis, Bertram, TX (US)

Correspondence Address: VINSON & ELKINS, L.L.P., 1001 FANNIN STREET, 2300 FIRST CITY TOWER, HOUSTON, TX, 77002-6760, US

(21) Appl. No.: 10/185,592

(22) Filed: Jun. 27, 2002

Related U.S. Application Data
(63) Continuation of application No. 09/755,628, filed on Jan. 5, 2001, now abandoned.

(51) Int. Cl. C12Q 1/68

(52) U.S. Cl. 435/6

(57) ABSTRACT

A method and associated compositions for the relative quantification of nucleic acid on an address-defined surface, involving fitting the nucleic acid with a generic oligonucleotide, and hybridizing the generic oligonucleotide with a directly or indirectly labeled complementary oligonucleotide. The method is applicable, for example, to SNP genotyping and gene expression analysis.

### BACKGROUND OF THE INVENTION

The present invention relates to the relative quantification of attached nucleic acids, and in particular to SNP genotyping and other applications where the relative quantification of attached nucleic acids is involved.

A large number of studies have shown an association between genetic variation and phenotype manifestation. To determine the genetic variations, many different methods of genotyping have been developed.

A Single Nucleotide Polymorphism (SNP) is a single nucleotide alteration or difference at specific loci among different individuals. It represents one of the most frequent and stable genetic variations. SNP genotyping, therefore, can be employed to provide genetic and physical maps of chromosomes to a very fine level of detail.

With the completion of the Human Genome Project and the development of high throughput DNA sequencing technology, SNP detection has been greatly accelerated. Many technology platforms have been commercially developed to detect SNP polymorphisms. Examples include the Cleavase-based Invader assay by Third Wave Technologies, single-base extension (SBE) and MALDI-TOF mass spectrometry by Sequenom, Taqman reaction-based assay by Perkin-Elmer, single base extensions based GBA (Genetic Bit Analysis) assay by Orchid, color coded microsphere and LabMap computer analysis-based assay by Luminex, Real-Time Sequencing-based assay by Pyrosequencing, oligonucleotide hybridization-based assay by Affymetrix, and SBE and fluorescence polarization-based assay by LJL BioSystems.

Among the variety of choices, only the Luminex color-coded bead and Affymetrix chip are designed for multiplex genotyping, in which multiple SNP sites are simultaneously genotyped in a single reaction. In exemplary multiplex genotyping, a color-coded bead or a physically defined location on a chip is attached with a SNP-specific oligonucleotide, which, in turn, is used for interrogating SNP genotypes of DNA samples (e.g., genomic or cDNA). The interrogation technique can be, for example, SNP-specific hybridization, the Oligonucleotide Ligation Assay (OLA) [see U.S. Pat. No. 4,883,750 to N. M. Whiteley et al., U. Landegren, et al., Science 241:1077 (1988), D. Y. Wu et al., Genomics 4:560 (1989), F. Barany, Proc. Nat'l Acad. Sci. USA, 88:189-193 (1991)], all of which are incorporated by reference herein in their entireties, or any other assay that can differentiate two alleles of a SNP.

The OLA is advantageous because it combines specificity of both hybridization and the enzymatic reaction of Taq ligase. In an OLA assay, a SNP allele-specific oligonucleotide (Capture oligonucleotide), having a sequence hybridizing to the 5′-upstream side of the target SNP plus one of the alternate SNP nucleotides, is covalently liked to a common oligonucleotide (Reporter oligonucleotide), having a sequence hybridizing immediately to the 3′ downstream side of the target SNP in a reaction catalyzed by Taq ligase. The reaction requires a perfect match between the Capture oligonucleotide and target DNA at the SNP site. Mismatches will abort the OLA reaction. (In this description, the terms oligonucleotide and oligo are used interchangeably.)

In this way, the two alleles of a SNP are differentiated. While the reaction requires a perfect match between the oligonucleotides and the target DNA around the SNP site, there is no such constraint for the oligonucleotide sequences 15 nucleotides or so upstream or downstream of the SNP site.

At present, OLA reactions are typically monitored by the fluorescent signal produced by a fluorescent label attached to the Reporter oligonucleotide at the specific address (or specific distinguishable bead) where the Capture oligonucleotide is located. The Reporter oligonucleotide can be directly labeled with a fluorescent label (e.g., fluorescein), or indirectly labeled, e.g., by attaching biotin to the oligonucleotide, and then staining with a strepavidin-phycoerythrin conjugate. The choice of the fluorogenic dye is, in part, determined by the wavelength of the excitation light generated by the genotyping equipment to be used. For example, current Luminex and Affymetrix instruments use a Yag or Argon laser to provide excitation light, at a wavelength where phycoerythrin is the brightest and most commonly used dye.

Though the OLA offers advantages for specificity, unfortunately, the fluor-labeled oligonucleotides are very expensive. Also, ordering such labeled oligonucleotides through a commercial source is very time-consuming, since each individual reporter oligonucleotide must be individually labeled. For chromosomal scanning or genetic linkage studies (as well as in other applications) hundreds or thousands of SNPs must be genotyped. Thus, the cost of individually labeling Reporter oligonucleotides is beyond the means of many researchers.

Recently, lannone et al. (2000) Cytometry 39:131-140, described OLA using short and degenerate 8-base (6 defined+2 degenerated) Reporters to replace perfectly matched 18-base oligonucleotides. They intended to use a limited set of oligos to replace the extremely large number that would otherwise be called for to cover all possible sequences of the Reporters. However, the scheme still requires 4⁶=4096 syntheses of specially labeled Reporter oligos, if the system is to be used for high throughput assays for a variety of different targets. Moreover, because only one in 16 of these degenerate oligos will be perfectly matched to the target and thus suitable for ligation, 15 unmatched oligos will remain in solution. In Iannone et al. supra, the unincorporated reporters did not appear to create problems, because the fluorescent dye, fluorescein, is a small molecule and is covalently bound to the Reporter oligo.

In contrast, phycoerythrin is a large protein (240 kD). Due to its large size, phycoerythrin can only be applied at a very low molar concentration. The limited number of phycoerythrin molecules can be readily saturated by the abundant unincorporated Reporter, which will greatly diminish the fluorescent signal on the beads to which Reporter is linked. Thus, the Iannone et al. supra, scheme is not applicable to the current Luminex and Affymetrix instruments. While the unincorporated Reporter can be removed mechanically by washing, the extra step is quite undesirable for high throughput genotyping, as it requires highly repetitive and precise pipetting, which is rather error-prone, especially where the reaction volumes are small.

### SUMMARY OF THE INVENTION

The methods of the present invention avoid cost and convenience limitations of present genotyping methods and materials, by dramatically reducing the numbers of different labeled oligonucleotides that will be needed to conduct genotyping assays or other determinations of the presence or amount of a specific nucleic acid sequence in a sample or assay. The method involves detecting and/or quantifying the label signal, e.g., the fluorescent signal, corresponding to bound Reporter oligonucleotides by fitting all Reporter oligonucleotides with a generic oligonucleotide sequence, and hybridizing the generic oligonucleotide with a labeled complementary generic oligonucleotide. This method can be readily incorporated in a large number of different configurations that are adapted for particular types of determinations, e.g., SNP genotyping.

The present methods and compositions are especially advantageous for multiplex determinations and/or conducting large numbers of assays, but are not limited to those applications.

Thus, in a first aspect, the invention provides a method for quantifying a specific nucleic acid sequence, e.g., in an assay or sample, by contacting at least one first oligonucleotide with at least one capture oligonucleotide under hybridization conditions. The first oligonucleotide is preferably PCR amplified genomic DNA (see R. K. Saiki, et al., Science 239:487 (1988) and Mullis, U.S. Pat. No. 4,683,202). Such a capture oligonucleotide will hybridize to a first oligonucleotide, and the 3′-terminal nucleotide of the capture oligonucleotide will be complementary to the corresponding nucleotide in the first oligonucleotide if the first nucleotide is a specified Target oligonucleotide, and will not be complementary if the first oligonucleotide is not the specified Target nucleotide. The method also involves contacting the first oligonucleotide with a corresponding Reporter oligonucleotide under hybridization conditions. A 5′-portion of the Reporter oligonucleotide at least 4 nucleotides in length (of length sufficient to provide hybridization to a complementary sequence under the hybridization conditions and support a ligation reaction) is complementary to the specific Target oligonucleotide. A 3′-portion of at least 4 nucleotides in length of the Reporter oligonucleotide is not complementary to the specified Target oligonucleotide. The Reporter oligonucleotide will hybridize to the specified Target oligonucleotide immediately adjacent to the Capture oligonucleotide. The first, Capture, and Reporter oligonucleotides are subjected to ligation conditions, in which the Capture oligonucleotide will be ligated to the Reporter oligonucleotide only if the 3′-terminal nucleotide is complementary to the corresponding nucleotide of the first oligonucleotide. The Reporter oligonucleotide is contacted with labeled oligonucleotide that will specifically hybridize to the 3′-portion of the Reporter oligonucleotide under hybridization conditions. Different Capture oligonucleotides ligated with Reporter oligonucleotides are attached at different distinguishable addresses, and the presence and/or amount of labeled oligonucleotide at one or a plurality of distinguishable addresses is determined as an indication of the presence or amount of specific Target oligonucleotide present.

In preferred embodiments, a plurality of different Reporter oligonucleotides are used, each including the same nucleotide sequence in the 3′-portion. This allows the use of a common, or generic labeled oligonucleotide.

Thus, in preferred embodiments, only one nucleotide sequence is used for the labeled oligonucleotide complementary to the 3′-portions of a plurality of different Reporter oligos.

In preferred embodiments, the determination is performed for a plurality of different Target oligonucleotides (also in other genotyping and presence, or quantity, determination methods described herein) in a single assay, and thus involves multiplex determinations. Alternatively, in preferred embodiments, the determinations of different Target oligos are performed on nucleic acid derived from the same organism, the same set or sets of organisms, are performed under the same contract or other agreement between two or more parties to perform such determinations, or are performed within a limited time period, e.g., one day, one week, or one month (though determinations may extend beyond such periods, in such embodiments a plurality of determinations are performed with such a specified time. Such a plurality of determinations, or plurality of different Target nucleic acid sequences may, for example, include at least 2, 3, 4, 5, 6, 8, 10, 20, 30, 40, 50, 70, 100, 200, 300, 400, 500, 1000, or more such determinations or targets.

In preferred embodiments involving a plurality of different Target oligonucleotides (including, for example, sequences including different SNP sites, sequences including alternative nucleotides at one or more SNP sites, sequences from different loci in a source sequence, and/or sequences from different sources), the determination also involves determining the respective numbers of the different Target oligonucleotides attached at a plurality of different distinguishable addresses. In this way, the presence and/or amount of different Target nucleic acids can be determined. Different Target nucleic acids can also be grouped, so that Target nucleic acids with a selected relationship or relationships are attached to the same distinguishable address.

Thus, in preferred embodiments, the respective numbers of different Target oligonucleotides attached at a plurality of different distinguishable addresses is indicative of the numbers or relative numbers of the respective different nucleotides present in at least one Single Nucleotide Polymorphism (SNP) site.

In a related aspect, the invention concerns a method for determining the quantity or presence of one or more Target nucleic acids in a sample by specifically associating a Reporter oligonucleotide(s) with Target nucleic acid from said sample. Each Reporter oligonucleotide includes a generic (i.e. common) oligonucleotide sequence that is not complementary to the Target nucleic acid. The method also involves hybridizing the generic oligonucleotide sequence with a labeled complementary oligonucleotide, and attaching the Target oligonucleotide at a distinguishable address. The presence of the labeled complementary oligonucleotide (generally the label itself) at the distinguishable address is indicative of the presence or amount of the Target nucleotide in the sample.

In preferred embodiments, the generic oligonucleotide sequence is at the 3′-end of the Reporter oligo. Preferably the generic sequence is at least 4, 6, 8, 10, 12, 15, 17, 20, or 30 nucleotides in length, preferably in a range specified by taking any of the listed lengths as a lower limit and any longer length as an upper limit. Limits may also be 35, 40, 45, or 50 nucleotides. Longer lengths may also be used.

In another related aspect, the invention provides a method for genotyping at least one SNP site in Target nucleic acid sequence from at least one organism. The method involves specifically hybridizing a Capture oligonucleotide to a Target nucleic acid sequence containing a SNP site, where the 3′-terminal nucleotide of the Capture oligonucleotide will be complementary to one of the alternate nucleotides at the SNP site, and hybridizing a Reporter oligonucleotide to the Target oligonucleotide immediately 3′ of the Capture oligonucleotide. The Reporter oligonucleotide also includes a 3′-portion of at least 4 nucleotides in length that does not hybridize to the Target oligonucleotide, preferably at least 5, 6, 7, 8, 9, 10, 12, 15 nucleotides in length. Preferably the 3′-portion is not more than 30, 20, 15, 12, or 10 nucleotides. In various embodiments, the length of the 3′-portion is in a range defined by taking any two of the lengths mentioned as inclusive endpoints for the range. The first or Target, Capture, and Reporter oligonucleotides are subjected to ligation conditions, where the Capture oligonucleotide will be ligated to the adjacent Reporter oligonucleotide only if the nucleotide at the SNP site is complementary to the 3′-terminal nucleotide of the Capture oligonucleotide. Reporter oligonucleotide is also contacted with a labeled oligonucleotide that will specifically hybridize to the 3′-portion of the Reporter oligonucleotide under hybridization conditions. Capture oligonucleotide ligated with Reporter oligonucleotide is attached at the distinguishable address, such that different Capture/Reporter oligos will be attached at different addresses. Determining whether the labeled oligonucleotide is present at a particular distinguishable address indicates the genotype of the Target nucleic acid sequence at the SNP site. That correlation is present because only ligated Capture/Reporter, corresponding to a particular SNP variant at a particular SNP site, will attach label at an address.

Preferably the at least one SNP site is a plurality of SNP sites, e.g., at least 2, 3, 4, 5, 6, 7, 8, 9, 10, 20, 30, 40, or more SNP sites.

Preferably the genotyping includes determination of the presence of alternate nucleotides at least one SNP site, preferably at a plurality of SNP sites, e.g., a number of sites as described herein.

In keeping with the aspects above, the invention also concerns complexes of oligonucleotides. Thus, in another aspect, the invention includes at least one complex of associated oligonucleotides, where each such complex includes a Target oligonucleotide, with a Capture oligonucleotide and a Reporter oligonucleotide hybridized to it. The Capture oligonucleotide and Reporter oligonucleotide are hybridized to immediately adjacent positions on the Target oligonucleotide, and the 3′-end of the Reporter oligonucleotide is not hybridized to said Target oligonucleotide. Instead, a labeled oligonucleotide is hybridized to the 3′-end of the Reporter oligonucleotide.

Preferably the Capture oligonucleotide and the Reporter oligonucleotide are ligated together. Thus, the ligated Capture and Reporter oligonucleotides form a longer oligonucleotide.

In preferred embodiments, the complex is in an assay solution, e.g., as will be formed in methods described above or otherwise described herein. Also in preferred embodiments, the complex is attached to a solid phase surface at a distinguishable address. The composition having that solid phase surface may, for example, be in suspension in an assay solution, or may be a chip or plate.

In preferred embodiments, there are a plurality of complexes in a single solution or on a single solid phase surface. The plurality of complexes includes a plurality of different Target oligonucleotides, a plurality of different Capture oligonucleotides, and a plurality of different Reporter oligonucleotides, where the different Reporter oligonucleotides have the same nucleotide sequence hybridized to labeled oligonucleotide.

In a related aspect, the invention also provides at least one complex of associated oligonucleotides. Each such complex includes a Target oligonucleotide, and a Reporter oligonucleotide specifically hybridized to the Target oligonucleotide, where a terminal portion at least 4 nucleotides in length of the Reporter oligonucleotide is not hybridized to the Target oligonucleotide. The complex also includes a labeled oligonucleotide hybridized to the terminal portion of the Reporter oligonucleotide.

In preferred embodiments there are a plurality of such complexes in a single solution or on a single solid phase surface. The plurality of complexes includes a plurality of different Target oligonucleotides, and a plurality of different Reporter oligonucleotides. Each of the different Reporter oligonucleotides has the same nucleotide sequence in the terminal portion.

Preferably such a complex(es) is attached to a solid phase surface at a distinguishable address.

Likewise, in another aspect, the present invention provides a kit for genotyping at least one SNP site in a nucleic acid from an organism. The kit includes at least one solid phase surface with distinguishable address, The solid phase surface has a chemical entity that will bind a Capture oligonucleotide under binding conditions. Such a chemical entity can, for example, be a nucleotide sequence or a member of a specific binding pair, such as one of an antibody or corresponding antigen, or avidin or strepavidin. The kit also includes at least one Capture oligonucleotide, that includes a nucleotide sequence selected to hybridize to potential Target nucleotide sequence (e.g., in a Target oligonucleotide). The kit also includes at least one Reporter oligonucleotide that includes a nucleotide sequence selected to hybridize to a potential Target nucleotide sequence (the same target sequence as for the Capture oligonucleotide) immediately 3′ of the Capture oligonucleotide. The Reporter oligonucleotide also includes a 3′ nucleotide sequence that does not hybridize to the target. For kits that contain a plurality of different Reporter oligonucleotides, a plurality (and preferably all) of the different Reporter oligonucleotides contain the same 3′ sequence that does not hybridize to Target nucleic acid. Further, the kit includes a labeled oligonucleotide that will hybridize to the 3′-portion of the Reporter oligonucleotide under hybridization conditions.

In preferred embodiments, the kit also contains a ligase that, under selective ligation conditions, will not ligate adjacent Capture and Reporter oligonucleotides hybridized to template nucleic acid if the 3′-terminal nucleotide of the Capture oligonucleotide is not complementary to the corresponding nucleotide of the template nucleic acid.

In preferred embodiments, the kit contains an attachment oligonucleotide that includes a sequence complementary to a 5′-portion of the Capture oligonucleotide, where the attachment oligonucleotide is attached to a distinguishable address on a solid phase surface.

In yet another aspect, the invention provides a kit for detecting the presence and/or amount of at least one Target nucleic acid in a sample. The kit contains a labeled oligonucleotide, and written instructions describing a method for using the labeled oligonucleotide to determine the presence or amount of Target nucleic acid in a sample by specifically associating Reporter oligonucleotide with Target nucleic acid; hybridizing the labeled oligonucleotide to the Reporter oligonucleotide; attaching the Reporter oligonucleotide to a distinguishable address; and determining the label signal from the distinguishable address as an indication of the presence or amount of the Target nucleic acid in the sample.

In preferred embodiments, the kit includes a plurality of different Reporter oligonucleotides, each different Reporter oligonucleotide including a sequence complementary to the labeled oligonucleotide.

In preferred embodiments, the kit contains a plurality of different Capture oligonucleotides, wherein each different Capture oligonucleotide includes a sequence selected to bind to Target nucleic acid immediately adjacent to a particular Reporter oligonucleotide. Preferably the kit includes both a plurality of different Capture oligos and a plurality of different Reporter oligos. In kits adapted for SNP genotyping, preferably there is one Reporter oligonucleotide for a set of alternate Capture oligos for a particular SNP site. Preferably the set includes a Capture oligo for each alternate nucleotide known to be present at the SNP site, and may also include oligos for the other nucleotides, e.g., for use as controls. (Similarly for other SNP sites for which oligonucleotides in the kit are targeted.)

In preferred embodiments, the kit includes a DNA ligase, preferably a thermostable DNA ligase, such as Taq DNA ligase.

In still another aspect, the invention concerns a kit for determining the presence and/or amount of Target nucleic acid in a sample. The kit includes a plurality of different Reporter oligonucleotides, where each such different Reporter oligonucleotide includes a sequence selected to hybridize to Target nucleic acid and a sequence complementary to a common oligonucleotide. The kit also includes a labeled oligonucleotide that includes the sequence of the common oligonucleotide.

Preferably the kit also includes written instructions describing a method for using the labeled oligonucleotide and the Reporter oligonucleotide to determine the presence or amount of Target nucleic acid in a sample by specifically associating Reporter oligonucleotide with Target nucleic acid; hybridizing the labeled oligonucleotide to the Reporter oligonucleotide; attaching the Reporter oligonucleotide to a distinguishable address; and determining the signal from the distinguishable address as an indication of the presence or amount of the Target nucleic acid in the sample.

As used herein, the term “nucleic acid ” refers to a covalently linked chain of nucleotides (which may or may not also have other moieties or structures attached), and includes oligonucleotides and polynucleotides.

The term “oligonucleotide”, or equivalently “oligo”, is used to refer to nucleic acid molecules that include a sequence of 3-5000 covalently linked nucleotides. In preferred embodiments, a particular oligonucleotide has a length selected to be appropriate for its role in the particular application as understood by those practiced in the art. For example an oligonucleotide may contain 3-3000, 4-2000, 4-1000, 6-1000, 8-1000, 4-500, 6-500, 8-500, 10-500, 15-300, 15-200, or 15-100 covalently linked nucleotides.

As used in connection with the present methods, the term “generic oligonucleotide” refers to an oligonucleotide that is not required to have a specific sequence related to a nucleic acid being quantitated (i.e., Target nucleic acid or template). The sequence of the generic oligonucleotide may be selected to provide useful characteristics, however. For example, the generic oligonucleotide sequence may be chosen to have a melting point from a perfectly complementary sequence in a particular temperature range e.g., 50-60° C., and/or to avoid binding to a portion of a nucleic acid being quantitated, and/or to avoid binding to other nucleic acids in a reaction mixture.

In the context of this invention, the term “attached nucleic acid” refers to a nucleic acid that is attached in an address-specific (e.g., location-specific) manner to a solid phase surface, e.g., a particle, bead, plate, chip, or other solid surface. For example, the nucleic acid can be attached to a specific, distinguishable site in an array, e.g., on a glass or polystyrene slide or chip, or may be attached to a coded bead or other particle, e.g., a color coded bead. In such bead or particle embodiments, the coding of the bead or particle provides the specific identification in the same manner as provided by the specific location in an array. The attachment may be direct or indirect, and may involve covalent bonding, nucleic acid hybridization, or any other type of binding association sufficient to provide the address specific association.

In the various aspects and embodiments of the present invention, the organism, or source of nucleic acid being determined, first oligonucleotide, Target nucleic acid or oligonucleotide, or similar nucleic acid being assayed, can be from any source. For example, the organism or DNA source may be directly from an organism, or from cells derived from an organism, from nucleic acid derived from such a source, or synthetic nucleic acid. For example, without limitation, an organism or source may be a virus, bacterium, yeast, fungus, plant, vertebrate, invertebrate, crustacean, fish, bird, or mammal. Mammals can, for example, be human, ungulate such as bovine (e.g., cattle), porcine, sheep, ruminants, dogs, cats, rats, or mice.

Also in the various aspects and embodiments of the present invention involving distinguishable addresses, distinguishable addresses may be of various types. For example, the address may be a physical location on an array. Thus, the addressing can involve the attachment of an oligo(s) at a defined position(s) on such an array, e.g., a microarray. Similarly, distinguishable addresses may be provided by coded beads (e.g., polystyrene or latex microspheres) or particles. Thus, the addressing can involve attachment of an oligonucleotide to such a coded bead. The coding may be provided in various ways, e.g., by fluorescence color based on the relative amounts of two or more different colored fluorescent dyes attached or incorporated in the bead or particle, or by distinguishable combinations of other labels.

In preferred embodiments, the label on the labeled oligonucleotide is a fluorescent label, which can be directly or indirectly attached. However, other labels can be used as alternatives or even in combination, e.g., light scattering labels and radiolabels. Indirect labeling uses a binding moiety on the labeled oligo that attaches the detectable label. For example, the binding moiety can utilize a nucleotide sequence that provides binding by nucleic acid hybridization, antibody/antigen binding, avidin or strepavidin/biotin binding, or other binding pair interaction.

In preferred embodiments, Capture oligonucleotides are attached to the distinguishable address (e.g., addressable location(s)) using nucleic acid hybridization to an oligonucleotide (or different oligonucleotides) attached at the address(s).

In order to provide greater signal, in some embodiments of the methods described herein involving ligation of oligonucleotides, it can be advantageous to increase the number of ligated oligos relative to the number of Target nucleic acid sequences in an assay. Thus, in preferred embodiments, ligation conditions are repeated a plurality of times, preferably using thermal cycling to allow ligated oligos to be separated from template (i.e., Target nucleic acid) and new Capture and Reporter oligos to hybridize and be ligated. The process can be repeated a few (e.g., 2, 3, 4, 5, 6, 7, 8, 9, or 10) times, or more (e.g., up to 15, 20, 30, 40, 50, 60, 70, 80, 90, or 100 times, or even more). Thus, it is advantageous to use a thermostable DNA ligase, e.g., Taq DNA ligase.

In order to facilitate the assay, in preferred embodiments of the methods described herein, the number of potential specific Target oligonucleotides is increased by amplification. Thus, a desired nucleic acid sequence is amplified, e.g., using the PCR, before, during, or after the ligation portion of the assay.

Additional embodiments will be apparent from the following Detailed Description and from the claims.

### DETAILED DESCRIPTION OF THE PREFERRED EMBODIMENTS

The drawing will first be briefly described.

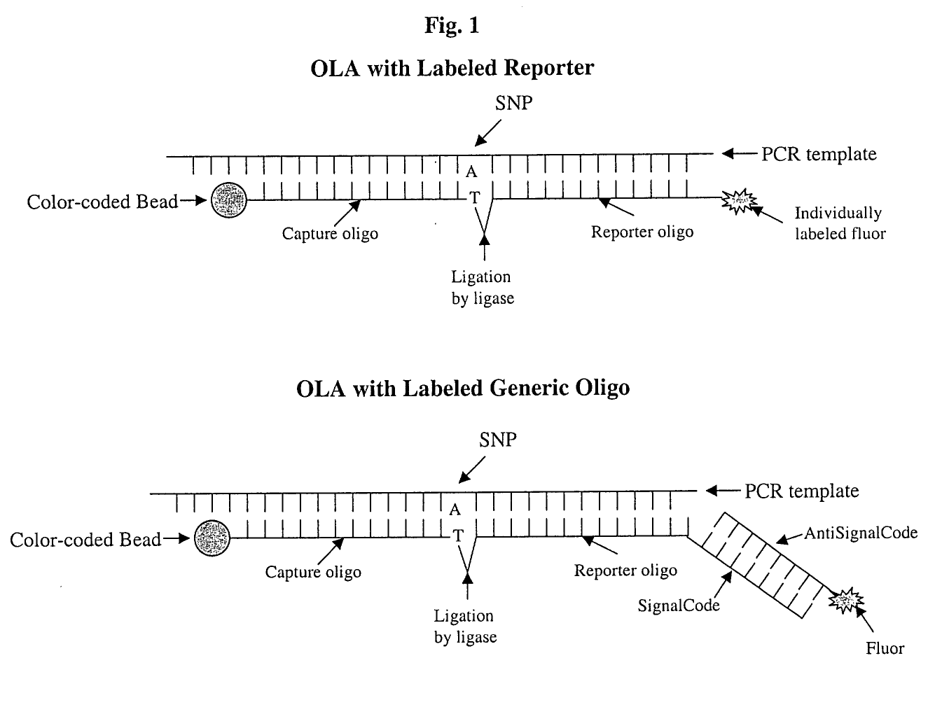

FIG. 1 includes two schematic diagrams of oligonucleotide ligation assays (OLA) for SNP genotyping. The top diagram illustrates conventional OLA using labeled Reporter oligonucleotides. The bottom diagram illustrates an embodiment of the present invention, in which a generic oligonucleotide hybridizing to the 5′-terminal portion of the Reporter oligonucleotide is used.

### INTRODUCTION

As pointed out in the Background, though the OLA is useful for SNP genotyping and other applications for identifying the presence of particular oligonucleotides, the large number of labeled oligonucleotides required for high throughput analyses present high costs in money and time. While providing some improvement, the method described in lannone et al. supra, still requires a large number of labeled oligos and is not readily applicable to current equipment.

Thus, the present methods are advantageous to avoid the high cost and lengthy time associated with producing such large number of fluorescent Reporter oligonucleotides by utilizing generic labeled oligonucleotides, such that the same one, or same few, labeled oligonucleotides can be used for all Target oligonucleotide analyses. Thus, the present methods are particularly desirable for high throughput genotyping, but are not limited to such uses.

The present invention can be set up in a large number of different configurations. The various embodiments have in common the use of a generic oligonucleotide (or a small set of generic oligos, e.g., 2, 3, 4 or other small number of different oligos) and hybridization of a complementary labeled oligo to the generic oligo.

For example, the Capture oligo can be attached to the distinguishable address directly or indirectly. In this context, direct attachment involves a binding interaction between the oligo (which can include a covalently attached linker) and the bead, chip, or other solid phase surface, and/or covalent bonding between the oligo and a moiety or functional group on the solid phase surface (e.g., a linker group). Indirect attachment involves attachment of the oligo to a solid phase surface through another (secondary) attachment molecule or molecules, where the association between the oligo and the secondary attachment molecule(s) is not, at least initially, covalent binding. For example, indirect attachment may utilize nucleic acid hybridization, antibody/antigen interaction, other binding pair interactions, as well as others.

Attachment to the distinguishable address can be done in a specific manner (corresponding to the Target). For example, where a Capture oligo is utilized, the Capture oligo can include a portion complementary to a nucleic acid sequence attached to the addressable surface. The attached nucleic acid sequence is different for each target sequence that it is desired to distinguish. Thus, the oligo on the addressable surface specifically pulls out a corresponding Capture oligo, and thus a corresponding Target molecule. Alternatively, the Capture oligo can be attached to the addressable surface in a non-specific manner. For example, a non-specific (i.e., generic) oligo can be attached to the surface. Target specific Capture oligos are then hybridized in an address-specific manner, such that a particular Capture probe and thus a particular Target will correspond to a particular address. In this manner, a single, or a few, attachment oligos can be utilized for many different Targets. Other types of molecular interactions (e.g., antigen/antibody) can also be used in similar specific or non-specific manner for attachment to the addressable surface.

The present invention is particularly advantageous as applied to the OLA. As indicated above, OLA involves ligation (e.g., using Taq DNA ligase) of Capture and Reporter oligonucleotides that are hybridized in adjacent positions to a Target nucleic acid molecule, generally an oligonucleotide. Generally the number of Target nucleic acid molecules is increased by amplification, e.g., using the Polymerase Chain Reaction (PCR), before the ligation reaction is carried out, in order to increase the detectability of the eventual signal. In the ligation reaction, the Capture and Reporter oligos will only be ligated if both are hybridized in adjacent positions, and the adjacent terminal nucleotides of both are complementary to the corresponding nucleotides of the Target. Mismatches may be created, for example, by the presence of a non-complementary nucleotide of a SNP at the terminal position of the Capture oligo.

In addition to the address-specific identification of Target, the OLA can also be used with size-based identification, as the ligation of Capture oligo and Reporter oligo provides a larger oligo. The size of the oligos can be size-separated using methods such as gel electrophoresis. Hybridization of the labeled oligo to the Reporter oligo provides a signal corresponding to the ligated oligos, thereby identifying (and quantitating if desired) the Target.

A schematic illustration of an exemplary use of the present invention for SNP genotyping, and a distinction from OLA that relies on labeled Reporter oligos is shown in FIG. 1. In this illustration, attachment to color-coded bead is used for the address specification. The “SignalCode” is a generic labeled oligonucleotide (fluor labeled).

The present invention is not limited to the use of the OLA. In other embodiments, the specificity to a Target nucleic acid molecule is provided by sequence specific hybridization. In such embodiments, the Target nucleic acids are fitted with the generic oligonucleotide by either direct ligation catalyzed by DNA ligase, by PCR using the generic oligonucleotide modified PCR primer, or any other method. Hybridization of the labeled reverse complementary oligo to be fitted to the generic oligo provides a signal corresponding to the Target nucleic acids, thereby identifying (and quantitating if desired) the Target.

In the various embodiments, preferably amplification is used to increase the number of Target molecules, e.g., using the PCR. However, if a sufficiently sensitive label/detection system is used, it can be possible to detect Target without amplification.

The present methods are applicable to many different organisms and compositions. For example, the present methods and compositions can be used for humans and other primates, ungulates such as cattle and other bovines, swine, and bacteria, among many others.

### Oligonucleotide Synthesis

All the described oligonucleotides can be synthesized by convention synthesis methods, preferably using automated DNA synthesizers, e.g., by commercial oligonucleotide synthesis services . The basic chemistry of the automated DNA synthesis is the consecutive removal and addition of sugar-protecting groups. With the first nucleotide being attached to a solid support, the synthesis begins as 5′ hydroxyl protection group dimethoxytrityl ether is removed by dichloroacetic acid in dichloromethane. After the deblocking, the hydroxyl becomes the only reactive nucleophile covalently coupled to the solid support. Next, highly reactive phosphoamidite modified nucleotide is simultaneously injected with the weak acid tetrazole. The nitrogen of the phosphoramidite becomes protonated and the phosphoramidite is easily attacked and replaced by the nucleophilic 5′ hydroxyl group. The reaction adds the second nucleotide to the first nucleotide. Repeating this cycle will lead to a stepwise, sequential addition of nucleotides to the growing oligonucleotide chain.

An amino group with a spacer, such as a C₁₂ spacer, can be fitted to the 5′ end of the oligonucleotide, e.g., Zipcode oligo, by many commercial oligonucleotide synthesis services. Phosphoramidite modified Amino C₁₂ is attached directly during oligonucleotide synthesis. It conjugates with high efficiency and does not typically require purification beyond standard desalting. Other amino modifiers can also be used, such as amino C₆ or Uni-link™, manufactured by CLONTECH Laboratories, Inc.

As indicated below, for an exemplary embodiment, the melting temperature (Tm) for each of the various oligonucleotides to be synthesized is selected to be approximately 55° C., although other temperatures can also be selected. The Tm of an oligonucleotide can be readily calculated using algorithms well-known to those familiar with nucleic acid hybridization assays. For example, the Tm for an oligonucleotide sequence can be calculated by any of a variety of computer programs, such as Oligo Analyzer freely available on the World Wide Web at the site idtdna.com, allowing the length of the oligonucleotide to be adjusted to provide the appropriate Tm.

### Hybridization Attachment Embodiment

In preferred embodiments of the invention, especially applicable to SNP genotyping, the method utilizes the OLA and attaches the Capture oligonucleotides (and thus also the Target, Reporter, and labeled oligonucleotides) to color-coded beads using nucleic acid hybridization. In these embodiments, four different types of oligonucleotides are utilized. (Such an exemplary embodiment is shown schematically in FIG. 1.) These are:
1. Address specific Zipcode oligonucleotides. The Zipcode sequences are preferably constructed of nucleotides selected to provide a Tm of about 55° C., e.g., in the range 50-60° C. (but not providing hybridization to the Target nucleotide(s). The 5′ ends of the Zipcodes are preferably substituted by an amino group, preferably with a C₁₂ linker (e.g., an alkyl linker), though a variety of other linkers can also be used. The amino group provides a reactive group for linking the Zipcode to a particle or surface, e.g., a color-coded particle from Luminex. The Zipcode oligonucleotides are attached to color-coded beads via a coupling reaction catalyzed by 1-ethyl-3-(3-dimethylaminopropyl)carbodiimide hydrochloride (EDC). The Luminex color-coded beads have been specially modified with a carboxyl group on their surface.

Carbodiimide catalyzes the formation of amide bonds between carboxylic acids and amines by activating carboxyl to form an O-urea derivative. This derivative reacts readily with nucleophiles, such as amine, to fit the Zipcode oligonucleotide on the surface of the beads. Use of Zipcode oligonucleotides or similar oligos is described in Barany et al., 1991, PNAS USA 88:189-193, and U.S. Pat. Nos. 6,027,889, 6,054,564, 5,830,711, and 5,494,810, as well as being utilized in Iannone et al., supra. All of these references are incorporated herein by reference in their entireties.
2. Capture oligonucleotides (complementary to a sequence on the 5′ side of the Target SNP plus one of the SNP alleles). The Capture oligos are also preferably designed to have a Tm of about 55° C., which can be readily achieved by adjusting the length of the oligonucleotides. The Capture oligonucleotides are fitted with “anti-Zipcodes” on their 5′ ends. The anti-Zipcodes are a set of oligonucleotides that are designed to bind to specific addresses by hybridizing to Zipcodes. The specific addresses can, for example, be color-coded beads or physically-defined locations on a solid phase surface.
3. Reporter oligonucleotides (complementary to a sequence on the 3′ side of the Target SNP). The Reporter oligos are fitted with one generic oligonucleotide, termed “Signalcode”, at their 3′ ends. The Signalcode is an oligonucleotide with a sequence preferably selected to have a Tm of about 55° C. and to not be complementary to the Target oligonucleotide, Zipcode, anti-Zipcode, Capture oligonucleotide, or Reporter oligonucleotide. The 5′ end is preferably substituted with a phosphate group, which facilitates the ligation reaction catalyzed by Taq ligase.
4. AntiSignalcode oligonucleotide. The anti-Signalcode oligo is complementary to the Signalcode sequence. Its 3′ or 5′ end is labeled, either directly or with an indirect label, e.g., a biotin that can be stained with a strepavidin-phycoerythrin conjugate.

As indicated in the oligonucleotide descriptions, all of the oligos are preferably designed to have Tm's of about 55° C., e.g., in the range 50-60° C. Other oligos can also be used that facilitate specific hybridization and/or the OLA reaction.

Preferably the Zipcode oligonucleotides are attached to color-coded beads, e.g., beads as provided by Luminex Corp. (Austin, Tx.). See, e.g., Fulton et al., 1997, Clin. Chem. 43:1749-1756; Kettman et al., 1998, Cytometry 33:234-243. Beads of those types can be distinguished by their fluorescence characteristics, e.g., by the specific combination of red and orange fluorescence (a fluorophore can then be used as an assay signal, e.g., a green fluorophore). Such color-coded beads can be coupled to the Zipcode oligos using a coupling reaction catalyzed by 1-ethyl-3-(3-dimethylaminopropyl) carbodiimide hydrochloride (EDC). The OLA is carried out in a reaction containing the Capture oligos, Reporter oligos, PCR DNA template (Target template), and Taq ligase. The consequent allele-specific concatenated oligonucleotides can be simultaneously sorted by Zipcoded bead and stained by a fluor- or biotin-labeled anti-Signalcode oligo in a single hybridization. The fluorescence of the stained bead can be measured on a flow cytometer along with the identification of the color-coded bead. The correlation of the fluorescence signal with the bead identification indicates which Target oligonucleotide(s) are present in the assay mixture.

Experiments such as those described below have repeatedly demonstrated the successful application of this embodiment for SNP genotyping. Such genotyping can also be confirmed by direct DNA sequencing or other genotyping methods. As indicated, the present method greatly reduces the cost of preparing various labeled Reporter oligos. By fitting a generic oligonucleotide Signalcode to each Reporter, one fluor- or biotin-labeled anti-Signalcode oligo is sufficient for all SNP genotyping.

Thus, the present invention provides a substantial improvement over prior OLA methods. The present invention not only reduces the number of fluor-labeled oligos to one, it also accommodates the most commonly used fluor, phycoerythrin. With the single anti-Signalcode oligo, strepavidin-phycoerythrin will not be saturated by the presence of abundant non-reactive degenerated biotinylated oligos, as would be the case with the Iannone et al. supra, method. The cost of fitting the Signalcode is relatively small compared to manufacturing specially labeled Reporter oligos, because the oligo synthesis process is highly automated, while the labeling reaction to produce labeled oligos requires much manual work.

The present invention utilizes the extensive knowledge that has developed on nucleic acid hybridization. Because oligonucleotide hybridization follows ideal second order kinetics, if one oligo concentration is kept constant (e.g., the labeled generic oligo), then hybridization is directly proportional to the concentration of its complementary strand (e.g., the Reporter oligo, and thus also the Target nucleic acid). The quantitative nature of the present invention indicates that it can be applied, not only to SNP genotyping and gene expression analysis, but also to any process that requires relative quantitation of attached nucleic acids.

### EXAMPLES

### Example 1

### Coupling of Zipcode to Beads

The Zipcode oligonucleotides were coupled to beads according to the following procedure. Disperse the beads in 100 μL of 0.1 M MES (pH 4.5). Add the amino-substituted oligonucleotide to a final concentration of 2 μM. Add 5 μL of freshly made EDC solution (1-ethyl-3-[3-dimethylaminopropyl]carbodiimide hydrochloride, 100 μg/μL). Incubate for 20 min at room temperature in the dark. Repeat the EDC addition and incubation. Wash the beads with 0.02% Tween 20 and then 0.1 % SDS. Resuspend the beads in TE buffer.

### Example 2

### Oligonucleotide Ligation Assay (OLA)

The OLA was carried out in a 20 μL reaction mixture containing 1× Taq ligase buffer, 0.5 pmol Capture oligo, 5.0 pmol Reporter oligo, 20 ng PCR SNP template, and 10 units of Taq ligase. The PCR SNP templates were generated from genomic DNA. Preferably the templates are 100-1000, bp in length, more preferably 150 to 1000 bp in length. It is generally more efficient to amplify small PCR targets. However, it may be difficult to measure PCR amplicon sizes by electrophoresis on an agarose gel when the amplicon size is less than 100 bp. If a different size determination technique is utilized that is suitable for shorter lengths, then smaller amplicon sizes may be preferred, for example, 20-100, 30-100, 30-80, or 40-80 bp. The reaction mixture was denatured at 96° C. for 2 min, followed by 55 cycles of 94° C. 15 sec, 37° C. 60 sec.

### Example 3

### SNP Detection

The sorting of oligonucleotides by Zipcoded bead and staining of Reporter by biotinylated anti-Signalcode oligo were carried out simultaneously in a single hybridization reaction. Fifty μL of hybridization mixture contains 1×TMAC buffer, 5000 Zipcoded beads for each SNP, 2.5 pmol biotinylated anti-Signalcode oligo, and 20 μL of OLA reaction mixture. The 1×TMAC buffer is 2.5 M TMAC (tetramethyl ammonium chloride), 0.15% SDS, 3 mM EDTA, and 75 mM Tris-HCl (pH 8.0). The reaction mixture was incubated at 95° C. for 5 min and then at 50° C. for 15 min.

The biotinylated anti-Signalcode oligos were stained with fluorescent strepavidin-phycoerythrin conjugate in a reaction containing 1×TE buffer and the conjugate at 10 μg/mL. The reaction was carried out at room temperature for 5 min. The beads were then measured for their fluorescent signal in a Luminex 100 flow cytometer.

### Example 4

### SNP Locus 1 Detection

In the this example, a bovine SNP site was amplified by a pair of PCR primers with sequences:

5′-CCTTTTCCTCTAGCATCAAGTTA-3′
and
5′-CAGACTGTGTGCTTCCTACAG-3′.

The PCR reaction mix contained 1×PCR reaction buffer, 300 μM dNTP, 300 nM PCR primers, 1.25 unit Taq DNA polymerase, and 100 ng genomic DNA in a volume of 50 μL. PCR amplification was performed with the following cycling parameter: 96° C. 2 min, then 35 cycles of 96° C. 30 sec, 55° C. 30 sec and 72° C. 1 min. The PCR product used for the OLA reaction. Three ZipCode oligonucleotides are:

5′-NH₂-GATGATCGACGAGACACTCTCGCCA-3′,
5′-NH₂-CGGTCGACGAGCTGCCGCGCAAGAT-3′
and
5′-NH₂-GACATTCGCGATCGCCGCCCGCTTT-3′.

The Zipcode oligonucleotides were coupled to beads according to the following procedure. Disperse the beads in 100 μL of 0.1 M MES (pH 4.5). Add the amino-substituted oligonucleotide to a final concentration of 2 μM. Add 5 μL of freshly made EDC solution (1-ethyl-3-[3-dimethylaminopropyl]carbodiimide hydrocloride, 100 μg/μL). Incubate for 20 min at room temperature in the dark. Repeat the EDC addition and incubation. Wash the beads with 0.02% Tween 20 and then 0.1% SDS. Resuspend the beads in TE buffer.

Three Capture oligonucleotides are:

5′-tggcgagagtgtctcgtcgatcatcCATCAAGTTAACACGTGG
AGC-3′,
5′-atcttgcgcggcagctcgtcgaccgCATCAAGTTAACACGTGG
AGG-3′
and
5′-aaagcgggcggcgatcgcgaatgtcCATCAAGTTAACACGTGG
AGW-3′.

In the Capture oligonucleotides, the lowercase sequences are antiZipcode sequences and the uppercase sequence is sequence complementary to the target sequence 5′ upstream of the SNP. The two nucleotides C and G at the 3′ ends correspond to the two alternate SNP nucleotides. The exemplary Signalcode Reporter oligonucleotide sequence is:

5′-phospho-ACATTCCCCAGTTTAATACTGCgtcaagatgctaccgtt
cag-3′.

The lowercase sequence is Signalcode and the uppercase sequence is the sequence complementary to the target sequence 3′ downstream of the target SNP. As a control, conventional Reporter oligonucleotide
5′-phospho-ACATTCCCCAGTTTAATACTGC-biotin-3′
   was also synthesized for a SNP genotyping assay.

The OLA was carried out in a 20 μL reaction containing: 1×Taq ligase buffer, 0.5 pmol of Capture oligo, 5.0 pmol of Reporter oligo (either Signalcode Reporter or conventional Reporter), 20 ng of PCR SNP template, and 10 units of Taq ligase. The PCR SNP templates were generated from genomic DNA. The acceptable size is from 150 bp to 1000 bp. The reaction mixture was denatured at 96 ° C. for 2 min and followed by 55 cycles of 94° C. 15 sec, 37° C. 60 sec.

The antiSignalcode is:

5′-ctgaacggtagcatcttgac-biotin-3′

which is reverse-complementary to the Signalcode of the SignalCode Reporter oligonucleotide. The sorting of oligonucleotides by Zipcoded bead and hybridization with biotinylated antiSignalcode oligo were carried out simultaneously in a single hybridization. Fifty microliters of hybridization mixture contains 1×TMAC buffer, 5000 Zipcoded beads for each SNP, 2.5 pmol of biotinylated antiSignalcode oligo, and 20 μL of OLA reaction mixture. 133 TMAC buffer comprises 2.5 M TMAC (tetramethyl ammonium chloride), 0.15% SDS, 3 mM EDTA and 75 mM Tris-HCl (pH 8.0). The reaction mixture was incubated at 95 ° C. for 5 min and then at 50 ° C. for 15 min. In the control experiment, the antiSignalcode was omitted for the conventional Reporter.

The biotinylated antiSignalcode oligos were stained with fluorescent strepavidin-phycoerythrin conjugate in a reaction containing 1×TE buffer and the conjugate of 10 μg/mL. The reaction was carried out at room temperature for 5 min. The beads were then measured for their fluorescent signal in a Luminex 100 flowcytometer. The following are the genotyping results with both Signalcode Reporter and conventional Reporter. The genotyping results were the same and were confirmed by direct DNA sequencing.

TABLE 1
Genotype with SignalCode Reporter

| Individual | 1 | 2 | 3 | 4 | 5 | 6 | 7 | 8 |
|---|---|---|---|---|---|---|---|---|
| C Bead | 408* | 280 | 60 | 293 | 355 | 356 | 252 | 399 |
| G Bead | 56 | 508 | 528 | 221 | 74 | 42 | 343 | 49 |
| A/T Bead | 32 | 37 | 32 | 28 | 33 | 31 | 29 | 27 |
| Genotype | C | C/G | G | C/G | C | C | C/G | C |

*relative fluorescent intensity

TABLE 2
Genotype with conventional Reporter

| Individual | 1 | d 2 | 3 | 4 | 5 | 6 | 7 | 8 |
|---|---|---|---|---|---|---|---|---|
| C Bead | 1293* | 768 | 60 | 1144 | 1208 | 1080 | 837 | 1240 |
| G Bead | 126 | 1073 | 1255 | 449 | 101 | 63 | 846 | 111 |
| A/T Bead | 55 | 41 | 33 | 38 | 38 | 32 | 34 | 44 |
| Genotype | C | C/G | G | C/G | C | C | C/G | C |

*relative fluorescent intensity

### Example 5

### SNP Locus 2 Detection

In this example, another bovine SNP site was amplified with a pair of PCR primers:

5′-AATAGTCATTTTGTCCAACCTCTA-3′
and
5′-CCTAAGCATTTTAGGTGAGATACA-3′.

The PCR was performed as described in Example 4.

Three Zipcode sequences are:

5′-NH₂-CGACTCCCTGTTTGTGATGGACCAC-3′,
5′-NH₂-CTTTTCCCGTCCGTCATCGCTCAAG-3′
and
5′-NH₂-GGCTGGGTCTACAGATCCCCAACTT-3′.

The Zipcode oligonucleotides were coupled to the Luminex color-coded bead according to the method described in Example 4.

Three Capture oligonucleotides are:

5′-gtggtccatcacaaacagggagtcgCAGGTAGGAAATTTGAAATG
TTA-3′,
5′-cttgagcgatgacggacgggaaaagCAGGTAGGAAATTTGAAATG
TTG-3′
and
5′-aagttggggatctgtagacccagccCAGGTAGGAAATTTGAAATG
TTY-3′.

The Signalcode Reporter oligonucleotide is:

5′-phospho-CAAGATTAAACTTTTAAAGTCACATGgtcaagatgctac
cgttcag-3′.

The conventional Reporter oligonucleotide is:

5′-phospho-CAAGATTAAACTTTTAAAGTCACATG-biotin-3′.

The OLA reaction was carried out as described in Example 4.

The antiSignalcode 5′-ctgaacggtagcatcttgac-biotin-3 is the same as in Example 4. The sorting of oligonucleotides by Zipcoded bead, hybridization of Reporter with biotinylated antiSignalcode oligo, and staining with phycoerythrin were carried out as described in Example 4. The following genotyping results were obtained:

TABLE 3
Genotype with SignalCode Reporter

| Individual | 1 | 2 | 3 | 4 | 5 | 6 | 7 | 8 |
|---|---|---|---|---|---|---|---|---|
| A Bead | 288* | 373 | 33 | 313 | 331 | 259 | 264 | 34 |
| G Bead | 32 | 36 | 328 | 35 | 33 | 27 | 24 | 511 |
| C/T Bead | 30 | 31 | 33 | 31 | 25 | 26 | 26 | 30 |
| Genotype | A | A | G | A | G | A | A | G |

*relative fluorescent intensity

TABLE 4
Genotype with conventional Reporter

| Individual | 1 | 2 | 3 | 4 | 5 | 6 | 7 | 8 |
|---|---|---|---|---|---|---|---|---|
| A Bead | 180* | 175 | 25 | 186 | 185 | 134 | 133 | 20 |
| G Bead | 22 | 22 | 206 | 27 | 24 | 19 | 22 | 240 |
| C/T Bead | 26 | 25 | 26 | 25 | 20 | 23 | 23 | 19 |
| Genotype | A | A | G | A | A | A | A | G |

*relative fluorescent intensity

Again the genotyping results are exactly the same with both methods.

| | | References |
|---|---|---|
| 6,027,889 | February 2000 | Barany et al . . . 435/6 |
| 6,054,564 | April 2000 | Barany et al . . . 536/22.1 |
| 4,883,750 | November 1989 | Whiteley et al . . . 436/6 |
| 5,830,711 | November 1998 | Barany et al . . . 435/91.1 |
| 4,683,202 | July 1987 | Mullis . . . 435/91.2 |

Cytometry v 39: 131-140 (2000) “Multiplexed single nucleotide polymorphism genotyping by oligonucleotide ligation and flow cytometry” Iannone et al.
Biotechniques v 28: 351-357 (2000) “New Cleavase Fragment Length Polymorphism method improves the mutation detection assay” Oldenburg et al.
Proc Natl Acad Sci USA. v 96: 10016-20 (1999) “Chip-based genotyping by mass spectrometry” Tang et al.
Genet Anal. v 14:143-149 (1999) “Allelic discrimination using fluorogenic probes and the 5′ nuclease assay” Livak
Genome Res. v 9: 167-174 (1999) “Mining SNPs from EST databases” Picoult-Newberg et al.
Genome Res. v 10: 1249-1258 (2000) “Determination of single-nucleotide polymorphisms by real-time pyrophosphate DNA sequencing” Alderborn et al.
Genome Res. v 10: 1126-1137 (2000) “Genome-wide detection of allelic imbalance using human SNPs and high-density DNA arrays” Mei et al.
Genome Res. v 9: 492-498 (1999) “Fluorescence polarization in homogeneous nucleic acid analysis” Chen et al.
Science v 239: 487-491 (1988) “Primer-directed enzymatic amplification of DNA with a thermostable DNA polymerase” Saiki et al.
Annu. Rev. Biophys. Bioeng. v 5: 337-361 (1976) “Hybridization and renaturation kinetics of nucleic acids” Wetmur
Science v 241: 1077-80 (1988) “A ligase-mediated gene detection technique” Landegren et al.
Genomics v 4:560-569 (1989) “The ligation amplification reaction (LAR)-amplification of specific DNA sequences using sequential rounds of template-dependent ligation” Wu et al.
Proc Natl Acad Sci USA. v 88:189-193 (1991) “Genetic disease detection and DNA amplification using cloned thermostable ligase” Barany
Applied Biosystems, 1985. User's Manual: Model 380B DNA synthesizer. Foster City, Calif.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted for use in genotyping particular nucleic acid segments and/or identifying the presence of a Target nucleic acid in a sample. The specific methods and compositions described herein as presently representative of preferred embodiments are exemplary and are not intended as limitations on the scope of the invention. Changes therein and other uses will occur to those skilled in the art which are encompassed within the spirit of the invention are defined by the scope of the claims.

It will be readily apparent to one skilled in the art that varying substitutions and modifications may be made to the invention disclosed herein without departing from the scope and spirit of the invention. For example, those skilled in the art will recognize that the invention may suitably be practiced using any of a variety of different oligonucleotides, buffers, labels, and solid phase surfaces.

The invention illustratively described herein suitably may be practiced in the absence of any element or elements, limitation or limitations which is not specifically disclosed herein as essential. Thus, for example, in each instance herein, in embodiments of the present invention, any of the terms “comprising,” “consisting essentially of” and “consisting of” may be replaced with either of the other two terms. The terms and expressions which have been employed are used as terms of description and not of limitation, and there is not intention, in the use of such terms and expressions, of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group. For example, if there are alternatives A, B, and C, all of the following possibilities are included: A separately, B separately, C separately, A and B, A and C, B and C, and A and B and C. Thus, the embodiments expressly include any subset or subgroup of those alternatives. While each such subset or subgroup could be listed separately, for the sake of brevity, such a listing is replaced by the present description.

While certain embodiments and examples have been used to describe the present invention, many variations are possible and are within the spirit and scope of the invention. Such variations will be apparent to those skilled in the art upon inspection of the specification and claims herein.

Other embodiments are within the following claims.

1. A method for determining the presence or amount of nucleic acid, comprising:
contacting at least one first oligonucleotide with at least one Capture oligonucleotide under hybridization conditions, wherein a said Capture oligonucleotide will hybridize to a said first oligonucleotide and the 3′-terminal nucleotide of said Capture oligonucleotide will be complementary to the corresponding nucleotide in said first oligonucleotide if said first nucleotide is a specified Target oligonucleotide, and will not be complementary if said first oligonucleotide is not said specified Target nucleotide;
contacting said first oligonucleotide with a Reporter oligonucleotide under hybridization conditions, wherein a 5′-portion of said Reporter oligonucleotide at least 4 nucleotides in length is perfectly complementary to said specific Target oligonucleotide and a 3′-portion at least 4 nucleotides in length of said Reporter oligonucleotide is not complementary to said specific Target oligonucleotide, and wherein said Reporter oligonucleotide will hybridize to said specific Target oligonucleotide immediately adjacent to said Capture oligonucleotide;
subjecting said first, Capture, and Reporter oligonucleotides to ligation conditions, wherein said Capture oligonucleotide will be ligated to said Reporter oligonucleotide only if said 3′-terminal nucleotide is complementary to the corresponding nucleotide of said first oligonucleotide;
contacting said Reporter oligonucleotide with labeled oligonucleotide that will specifically hybridize to said 3′-portion of said Reporter oligonucleotide under hybridization conditions;
attaching different Capture oligonucleotides ligated with Reporter oligonucleotides at different distinguishable addresses; and
determining whether said labeled oligonucleotide is present at a said distinguishable address as an indication of the presence or amount of said specific Target oligonucleotide.

2. The method of claim 1, wherein a plurality of different Reporter oligonucleotides are used, each including the same nucleotide sequence in said 3′-portion.

3. The method of claim 2, wherein only one nucleotide sequence is used for said labeled oligonucleotide complementary to said 3′-portion.

4. The method of claim 1, wherein said determining is performed for a plurality of different Target oligonucleotides.

5. The method of claim 4, wherein said determining further includes determining the respective numbers of said different Target oligonucleotides attached at a plurality of different distinguishable addresses.

6. The method of claim 5, wherein the respective numbers of said different Target oligonucleotides attached at said plurality of different distinguishable addresses is indicative of the relative numbers of respective different nucleotides present in at least one Single Nucleotide Polymorphism (SNP) site.

7. The method of claim 1, wherein said oligonucleotide is attached on an array.

8. The method of claim 1, wherein said oligonucleotide is attached to a coded bead.

9. The method of claim 1, wherein the label on said labeled oligonucleotide is a fluorescent label.

10. The method of claim 1, wherein the label on said labeled oligonucleotide is a radiolabel.

11. The method of claim 1, wherein the label on said labeled oligonucleotide is a light scattering label.

12. The method of claim 1, wherein the label on said labeled oligonucleotide is indirectly labeled.

13. The method of claim 1, wherein said Capture oligonucleotide is attached to said addressable location using nucleic acid hybridization to an oligonucleotide attached at said address.

14. The method of claim 1, wherein said ligation conditions are repeated a plurality of times using thermal cycling.

15. The method of claim 14, wherein said ligation conditions include the use of Taq DNA ligase.

16. The method of claim 1, wherein the number of potential specified Target oligonucleotides is increased by amplification.

17. A method for determining the quantity or presence of Target nucleic acid in a sample, comprising
specifically associating a Reporter oligonucleotide with said Target nucleic acid from said sample, wherein said Reporter oligonucleotide includes a generic oligonucleotide sequence that is not complementary to said Target nucleic acid;
hybridizing said generic oligonucleotide sequence with a labeled complementary oligonucleotide; and
attaching said Target oligonucleotide at a distinguishable address,
wherein the presence of said labeled complementary oligonucleotide at said distinguishable address is indicative of the presence or amount of said Target nucleotide in said sample.

18. The method of claim 17, wherein the label on said labeled oligonucleotide is a fluorescent label.

19. The method of claim 17, wherein the label on said labeled oligonucleotide is a light scattering label.

20. The method of claim 17, wherein said labeled oligonucleotide involves indirectly labeling.

21. The method of claim 20, wherein said indirect labeling utilizes strepavidin/biotin binding.

22. A method for genotyping at least one SNP site in Target nucleic acid sequence from at least one organism, comprising
specifically hybridizing a Capture oligonucleotide to a said Target nucleic acid sequence containing a SNP site, wherein the 3′-terminal nucleotide of said Capture oligonucleotide will be complementary to one of the alternate nucleotides at said SNP site;
hybridizing a Reporter oligonucleotide to said Target nucleic acid immediately 3′of said Capture oligonucleotide, wherein said Reporter oligonucleotide also comprises a 3′-portion at least 4 nucleotides in length that does not hybridize to said Target oligonucleotide;
subjecting said Target nucleic acid, Capture, and Reporter oligonucleotides to ligation conditions, wherein said Capture oligonucleotide will be ligated to said Reporter oligonucleotide only if the nucleotide at said SNP site is complementary to the 3′-terminal nucleotide of said Capture oligonucleotide;
contacting said Reporter oligonucleotide with a labeled oligonucleotide that will specifically hybridize to said 3′-portion of said Reporter oligonucleotide under hybridization conditions;
attaching Capture oligonucleotide ligated with Reporter oligonucleotide at said distinguishable address; and
determining whether said labeled oligonucleotide is present at said distinguishable address as an indication of the genotype of said Target nucleic acid sequence at said SNP site.

23. The method of claim 22, wherein said ligation conditions are repeated a plurality of times using thermal cycling.

24. The method of claim 23, wherein said ligation conditions include the use of Taq DNA ligase.

25. The method of claim 22, wherein said at least one SNP site is a plurality of SNP sites.

26. The method of claim 25, wherein said plurality of SNP sites is at least 5 SNP sites.

27. The method of claim 22, wherein said genotyping includes determination of the presence of alternate nucleotides in at least one SNP site.

28. The method of claim 22, wherein said organism is a mammal.

29. The method of claim 28, wherein said mammal is human.

30. The method of claim 28, wherein said mammal is bovine.

31. The method of claim 28, wherein said mammal is porcine.

32. The method of claim 28, wherein said mammal is a sheep.

33. The method of claim 22, wherein said organism is a bacterium.

34. The method of claim 28, wherein said organism is a plant.

35. At least one complex of associated oligonucleotides, each said complex comprising a Target oligonucleotide, having hybridized thereto a Capture oligonucleotide and a Reporter oligonucleotide, wherein said Capture oligonucleotide and said Reporter oligonucleotide are hybridized to immediately adjacent positions on said Target oligonucleotide and the 3′-end of said Reporter oligonucleotide is not hybridized to said Target oligonucleotide; and
a labeled oligonucleotide hybridized to said 3′-end of said Reporter oligonucleotide.

36. The complex of claim 35, wherein said Capture oligonucleotide and said Reporter oligonucleotide are ligated together.

37. The complex of claim 35, wherein said complex is in an assay solution.

38. The complex of claim 35, wherein said complex is attached to a solid phase surface at a distinguishable address.

39. The complex of claim 35, wherein said at least one complex is a plurality of complexes in a single solution, comprising
a plurality of different Target oiigonucleotides;
a plurality of different Capture oligonucleotides and
a plurality of different Reporter oligonucleotides, wherein said different Reporter oligonucleotides have the same nucleotide sequence hybridized to said labeled oligonucleotide.

40. At least one complex of associated oligonucleotides, each said complex comprising
a Target oligonucleotide;
a Reporter oligonucleotide specifically hybridized to said Target oligonucleotide, wherein a terminal portion at least 4 nucleotides in length of said Reporter oligonucleotide is not hybridized to said Target oligonucleotide; and
a labeled oligonucleotide hybridized to said terminal portion of said Reporter oligonucleotide.

41. The complex of claim 40, wherein said at least one complex is a plurality of complexes in a single solution, comprising
a plurality of different Target oligonucleotides; and
a plurality of different Reporter oligonucleotides, wherein said different Reporter oligonucleotides have the same nucleotide sequence in said terminal portion.

42. The complex of claim 40, wherein said complex is attached to a solid phase surface at a distinguishable address.

43. A kit for genotyping at least one SNP site in nucleic acid from an organism, comprising
at least one solid phase surface with distinguishable address, comprising a chemical entity that will bind a Capture oligonucleotide under binding conditions;
at least one said Capture oligonucleotide including a nucleotide sequence selected to hybridize to potential Target oligonucleotide;
at least one Reporter oligonucleotide including a nucleotide sequence selected to hybridize to a said potential Target oligonucleotide immediately 3′of said Capture oligonucleotide; and
a labeled oligonucleotide that will hybridize to a 3′-portion of said Reporter oligonucleotide under hybridization conditions.

44. The kit of claim 43, further comprising a ligase that, under selective ligation conditions, will not ligate adjacent Capture and Reporter oligonucleotides hybridized to template nucleic acid if the 3′-terminal nucleotide of said Capture oligonucleotide is not complementary to the corresponding nucleotide of said template nucleic acid.

45. The kit of claim 43, further comprising an attachment oligonucleotide comprising a sequence complementary to a 5′-portion of said Capture oligonucleotide, wherein said attachment oligonucleotide is attached to said solid phase surface.

46. A kit for determining the presence of at least one Target nucleic acid in a sample, comprising
a labeled oligonucleotide; and
written instructions describing a method for using said labeled oligonucleotide to determine the presence or amount of Target nucleic acid in a sample by specifically associating Reporter oligonucleotide with Target nucleic acid; hybridizing said labeled oligonucleotide to said Reporter oligonucleotide; attaching said Reporter oligonucleotide to a distinguishable address; and determining the signal from said distinguishable address as an indication of the presence or amount of said Target nucleic acid in said sample.

47. The kit of claim 46, further comprising a plurality of different Reporter oligonucleotides, each different Reporter oligonucleotides including a sequence complementary to said labeled oligonucleotide.

48. The kit of claim 47, further comprising a plurality of different Capture oligonucleotides, wherein each different Capture oligonucleotide includes a sequence selected to bind to Target nucleic acid immediately adjacent to a said Reporter oligonucleotide.

49. The kit of claim 48, further comprising a DNA ligase.

50. A kit for determining the presence of Target nucleic acid in a sample, comprising
a plurality of different Reporter oligonucleotides, each said different Reporter oligonucleotides comprising a sequence selected to hybridize to Target nucleic acid and a sequence complementary to a common oligonucleotide; and
a labeled oligonucleotide comprising the sequence of said common oligonucleotide.

51. The kit of claim 50, further comprising written instructions describing a method for using said labeled oligonucleotide and said Reporter oligonucleotide to determine the presence or amount of Target nucleic acid in a sample by specifically associating Reporter oligonucleotide with Target nucleic acid; hybridizing said labeled oligonucleotide to said Reporter oligonucleotide; attaching said Reporter oligonucleotide to a distinguishable address; and determining the signal from said distinguishable address as an indication of the presence or amount of said Target nucleic acid in said sample.
